(19) United States
(12) Patent Application Publication
(10) Pub. No.: US 2016/0324561 A1
(43) Pub. Date: Nov. 10, 2016

(54) Device for Locating an Entry Point of a Pedicle of a Vertebra

(71) Applicant: SAFE ORTHOPAEDICS, Eragny sur Oise (FR)

(72) Inventor: Herve Vouaillat, Venon (FR)

(21) Appl. No.: 15/110,490

(22) PCT Filed: Jan. 9, 2015
(86) PCT No.: PCT/FR2015/050045

§ 371 Date: Jul. 8, 2016

(30) Foreign Application Priority Data
Jan. 10, 2014 (FR) 1450164

(51) Int. Cl. A61B 17/88

(52) U.S. Cl. CPC A61B 17/8875; A61B 2017/90

(57) ABSTRACT

A location device for locating an entry point of a pedicle of a vertebra intended for being used with an instrument for perforating the pedicle, the location device comprising: an elongate aiming body having two end openings connected to one another by a guiding channel defining an axis for longitudinal guiding of the perforating instrument, the aiming body being extended at one of the ends by a fork overlapping the transverse process supported by the pedicle to perforate, the overlapping fork comprising two extensions defining a receiving area for the transverse process supported by the pedicle to be perforated, the receiving area having a first contact surface, configured to engage with an area at the junction of the transverse process and an articular process associated with the pedicle to be perforated, placed offset relative to the guiding axis.

### BACKGROUND

The invention relates to spinal surgery and, in particular, the implant of systems of pedicle screws by posterior approach.

More specifically, the invention relates to a device for locating an entry point of a pedicle of a vertebra intended for the implant of a pedicle screw through the pedicle.

The location device is mainly designed to locate the entry point of a pedicle of a lumbar vertebra.

The implant of a pedicle screw through a pedicle is carried out by posterior approach. Pilot holes are made in the pedicle before the implant of the pedicle screw through the pedicle.

Whether it consists of the perforation of pilot holes or even the implant of the pedicle screw, these operations are commonly carried out without direct visual control of the pedicle and the surrounding structures (spinal cord, vessels, nerves). But the pedicle, representing the most solid part of the vertebra in which a pedicle screw can be implanted, has a limited diameter, making its location and its perforation difficult. The location and the perforation of the pedicle are especially difficult in subjects of small size.

Now, the implant of screws not strictly in the pedicle, in particular implants that are too deep or internal may be the cause of more or less serious, sometimes irreversible neurological complications. In particular, beside the risks related to the deterioration of the surrounding structures such as the spinal cord or the vertebral artery, other complications may appear due to the poor position of the pedicle screw.

The location of a suitable entry point of a pedicle for the implant of a pedicle screw is therefore an especially difficult and tricky although crucial surgical procedure. The surgeon needs to have a good knowledge of the anatomy of the region in which the screw will be implanted and extensive experience.

In order to help the surgeon determine the entry points of pedicles, 3D systems of navigation are currently extensively used. Although these systems provide comfort and increase the precision, the associated equipment is cumbersome and difficult to use. Radioscopy is another technique commonly used. The main disadvantage, besides the cost, is the quantity of radiation to which the patient is exposed.

Confronted with these difficult and expensive techniques, specific aiming instruments have been developed. For example, we are familiar with American patent US2004/0073229 relating to an instrument to locate an entry point in the pedicle of a thoracic vertebra and guide a device for the perforation of said pedicle in given direction from a targeted entry point. More specifically, the instrument comprises a locating unit connected to a grip part by a rod body. The locating unit comprises a recess opening able to house the lower part of the blade associated with the pedicle to perforate, a locating fork to come into position on the pedicle to perforate and a conduit to receive the drilling device. The conduit is arranged with respect to the locating fork so as to present a centre positioned on a line extending from the centre of the locating fork. Therefore, when the locating fork is inserted on the pedicle and the hollowed part assembled on the arch, the conduit is automatically positioned and centred on the top part of the pedicle. The central position of the pedicle is thereby automatically located. When the perforation device is inserted in the conduit to perforate the pedicle, it is positioned on the top part of the pedicle to drill a hole according to a correct angle and thereby allow for the insertion of the pedicle screw in a satisfactory position.

However, the device in the aforementioned patent has several disadvantages. In particular, the configuration of the locating unit and the arrangement of the elements comprising it require the “cleaning” of the vertebra around the pedicle to perforate in order to have access to the pedicle. The use of the instrument thereby involves a relatively invasive surgical intervention. In addition, the use of the instrument requires the removal of a part of the vertebra for the placement of the locating unit at the pedicle. Moreover, in view of the configuration of the instrument, and in particular that of the locating fork, the instrument in the aforementioned patent can't be used on lumbar vertebrae, since access to the pedicle is prevented by the presence of lamina extending in a distinctly vertical plane. Moreover, it turns out that when the instrument is placed on the vertebra, one of the branches of the locating fork slightly extends in the spinal canal housing the spinal cord. Thereby, the use and handling of the device is relatively risky in view of the sensitive areas it comes into contact with.

The invention aims at correcting these problems. It proposes a location device to quickly and precisely locate the entry point of a pedicle to be perforated in view of the placement of a pedicle screw and guide a perforation instrument according to an optimum path avoiding all deterioration of the surrounding structures of the pedicle.

The invention also proposes a location device that can be used directly on a lumbar vertebra without the prior preparation of the vertebra or, when required by the anatomy of the patient, with minimum preparation. “Preparation of the vertebra” in particular refers to the removal of parts of the vertebra required to locate the pedicle to receive the pedicle screw.

The invention also proposes a location device that is not very invasive, is easy to use by the operator and doesn't involve any risks for the patient.

### SUMMARY

The invention relates to a device for locating an entry point of a pedicle of a vertebra intended for being used with an instrument for perforating the pedicle, the location device comprising an elongate aiming body, having two end openings connected to one another by a guiding channel defining an axis for longitudinally guiding the perforating instrument, said aiming body being extended at one of the ends by a fork overlapping the transverse process supported by the pedicle to be perforated, the overlapping fork comprising two extensions defining a receiving area for the transverse process supported by the pedicle to be perforated, said area having a first contact surface intended for engaging with an area at the junction of the transverse process and the articular process associated with the pedicle to be perforated, placed offset relative to the guiding axis.

Due to the arrangement of the overlapping fork and, in particular the offset position of the surface in contact with the axis of the guiding channel, the entry point of the pedicle to perforate for the placement of a pedicle screw is quickly and precisely located. The fork overlapping the traverse process simply has to be placed at the contact surface coming up against the junction of the transverse process and the pedicle. Once positioned, the location device of an entry point is such that the guiding channel is placed along an optimum pathway for screwing and reaches the pedicle by a perforation instrument avoiding any deterioration of the surrounding structures. The perforation instrument can then be introduced in the channel and moved until it reaches and perforates the pedicle at a given point.

Advantageously, the overlapping fork has a portion connecting the aiming body and the arranged extensions to determine a second contact surface that can come into contact with the articular process when the first contact surface is in contact with the area at the junction of the transverse process and the articular process.

Advantageously, the extensions are arranged at a distance from each other to enable the movement of the overlapping fork along the process to the junction between the transverse process and the pedicle to indicate.

Advantageously, the guiding axis of the guiding channel is parallel the longitudinal axis of the aiming body.

Advantageously, the receiving area for the transverse process extends parallel to the guiding axis of the guiding channel.

Advantageously, the receiving area for the transverse process is U-shaped and the base forms the first contact surface.

Advantageously, the aiming body comprises, at the end opposite the end supporting the overlapping fork, an extending arm along a longitudinal axis cutting the guiding axis.

Advantageously, the longitudinal axis of the arm, the guiding axis of the guiding channel and the longitudinal axis of the aiming body are coplanar.

Advantageously, the longitudinal axis of the arm has an angular separation with the guiding axis ranging from 90 degrees to 150 degrees.

Advantageously, the arm is arranged to enable the connection of a removable handle at the aiming body. According to a variant of the invention, the arm may form a handle on the aiming body.

Advantageously, the aiming body and the overlapping fork are formed from a single piece.

Advantageously, the aiming device is disposable.

### BRIEF DESCRIPTION OF THE FIGURES

Other goals and advantages of the invention will appear in the following description, with reference to the appended drawings, in which:

FIG. 1 represents a schematic perspective view of a device for locating an entry point according to the invention;

FIG. 2 represents a longitudinal cross-section of the location device in FIG. 1 according to axis II-II;

FIG. 3 represents a lateral view of the location device in FIG. 1;

FIG. 4 represents a view of the location device in FIG. 1 in position on a vertebra and a perforation instrument.

### DETAILED DESCRIPTION

FIGS. 1 to 4 describe a device for locating (1) an entry point of a pedicle of a vertebra, said device is intended to be used with an instrument for the perforation of the pedicle. The location device (1) comprises an aiming body (2), a fork (3) overlapping a transverse process associated with the pedicle of the vertebra to perforate and an arm (4) to hold the location device.

In the mode of construction illustrated, the entire length of the cylindrical aiming body (2) has a channel (5) crossing it. The channel defines a longitudinal guiding axis (AA) of the perforating instrument. We then speak of a guiding channel (5). As seen below, the fact that the guiding channel (5) has a certain length has the advantage that it prevents the deviation of the perforating instrument during the guiding operation.

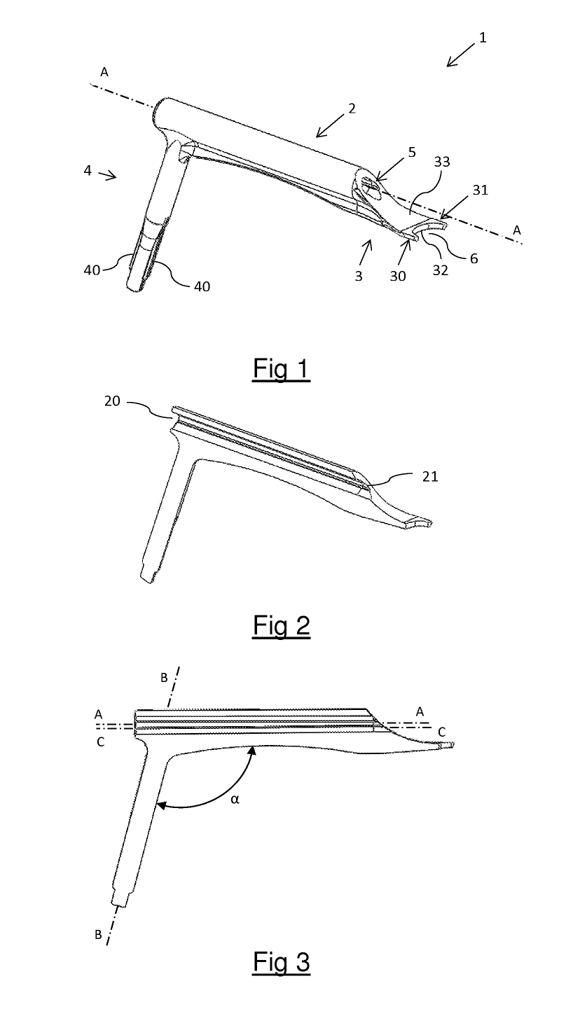

Advantageously, the guiding channel (5) is foreseen in the aiming body (2) so as to determine a guiding axis (AA) parallel to the longitudinal axis (CC) of the aiming body (2). In the mode of construction illustrated, the guiding channel (5) is arranged to present a guiding axis (AA) offset with respect to the longitudinal axis (CC) of the aiming body (2) in a radial direction opposite the arm (4) (FIG. 2). Of course, a guiding channel presenting a guiding axis coaxial with the longitudinal axis of the aiming body can be foreseen, without going beyond the scope of the invention.

The aiming body (2) is extended at one of its ends (21) by the overlapping fork (3). In the mode of construction illustrated, the overlapping fork (3) extends in a direction parallel to the guiding axis (AA). It also presents a nominal width greater than the diameter of the aiming body (2). It is of course obvious that the location device (1) may be equipped with an overlapping fork (3) presenting a different profile without going beyond the bounds of the invention.

The overlapping fork (3) is formed by two lateral extensions (30, 31) defining a receiving area (6) for the transverse process supported by the pedicle to perforate. Advantageously, the extensions (30, 31) are placed at a distance from each other so that the location device (1) can be easily placed on the transverse process and moved along the latter to reach the level of a groove located at the junction of the transverse process and the upper articular process, while providing a slight clearance between the location device (1) and the transverse process.

The overlapping fork (3) also presents a first contact surface (32) designed to come into contact with the bottom of the groove. As shown in FIG. 1, the inner contact surface (32) defines the bottom of the receiving area (6) of the transverse process. To ensure a certain stability of the location device (1) when it is in place on the transverse process, the first contact surface (32) is organised to present a form roughly complementary with that of the transverse process.

The overlapping fork (3) is arranged so that the inner contact surface (32) is located at a determined distance from the guiding axis (AA), i.e. not aligned on it. As will be clear below, the distance between the guiding axis (AA) and the inner contact surface (32) is determined so that, when the location device (1) is in position on the transverse process, with the inner contact surface (32) against the bottom of the groove, the perforation instrument inserted in the guiding channel (5) is placed in a position ensuring the aiming of an entry point of the pedicle.

The overlapping fork (3) comprises a portion connecting the aiming body (2) and the extensions (30, 31) arranged to determine a second contact surface (33) able to come into contact with one side of the upper articular process (then called articular facet) when the first contact surface (32) is in contact with the bottom of the groove. As illustrated in FIG. 1, the roughly flat, external contact surface (33) is located between the internal contact surface (32) and the opening of the outlet (21) of the guiding channel (5).

In addition, the aiming body (2), at the end opposite the end supporting the overlapping fork (3), comprises an arm (4), from an axis (BB) cutting the guiding axis (AA), said arm allowing for the grip of the location device (1). In the illustrated mode of construction, the grip of the location device (1) is obtained by a removable handle (not represented) that can be assembled on said arm (4). In this case, the arm (4) forming a connection arm for a means of gripping the aiming body (2) advantageously comprises longitudinal assembly ribs.

The arm (4) is arranged so that its axis, the guiding axis (AA) and the longitudinal axis of the aiming body (2) are coplanar.

Advantageously, the arm (4) has an angular offset with the aiming body (2) and thereby with the guiding channel (5) ranging from 90 degrees to 150 degrees. This facilitates the handing of the location device (1) to suitably place the overlapping fork (3) on the transverse process. In the illustrated example, the angular offset is about 120 degrees. This slope of the arm (4) with respect to the aiming body (2) naturally helps provide a good position for the guiding channel (5) with respect to the transverse process so as to guarantee the aiming of the pedicle.

Advantageously, the arm (4) and the overlapping fork (3) are formed from one piece with the aiming body (2). It is of course obvious that the invention is not limited to such a configuration and that a location device (1) where the arm (4) and/or the overlapping fork (3) are made of separate parts, able to be fixed on and removed from the aiming body (2).

Advantageously, the location device (1) is disposable. For this purpose, it is advantageously made of polymer.

Moreover, as indicated above, the arm (4) is configured in the illustrated mode of construction to provide for the fixation of a handle on the location device (1). According to a variant in the construction, the arm (4) may also be configured so as to directly form the handle of the aiming body (2). The arm (4) may also be assembled on and removed from the body. The arrangement of such an arm (4) will then be similar to the connecting arm described above.

According to another variant of the construction, the location device may not contain an arm. The location device is then held.

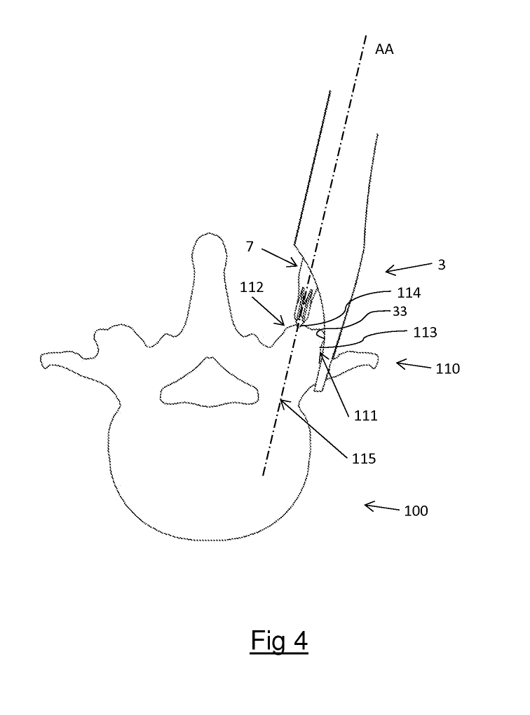

FIG. 4 shows the location device (1) in position on one of the traverse processes (110) of a vertebra (100). The location device is placed on the vertebra (100) as follows:

Location device (1) is first placed on the transverse process (110), the fork (3) overlapping the latter. The location device (1) is then moved along the transverse process (110) in the direction of the groove (111) located at the junction between the transverse process (110) and the upper articular process (112) associated with the pedicle (115) to perforate, until reaching said groove (111). Once the first contact surface (32) of the overlapping fork (3) is in contact with the bottom of the groove (111), the location device (1) is tipped so as to place the second contact surface (33) against the articular facet (113). The location device (1) is then in position to allow for a suitable location of the entry point (114) of the pedicle (115).

The perforation instrument (7) is then inserted in the guiding channel (5) and moved along the opening (20) of said guiding channel (5) to the outlet (21) of the latter. The movement of the perforation instrument (7) in the guiding channel (5) is continued until reaching the entry point (114) of the pedicle (115). The entry point (114) of the pedicle (115) is automatically reached by the perforation instrument (7) from the offset position of the overlapping fork (3) with the guiding channel (5) and, more specifically, the offset between the bottom of the receiving area (6) forming the first contact surface and the guiding axis of the guiding channel (5).

The invention is described in the above by way of example. It is understood that a person skilled in the art is able to make different variants of the invention without going beyond the scope of the invention.

1-11. (canceled)

12. A location device for locating an entry point of a pedicle of a vertebra intended for being used with an instrument for perforating the pedicle, the location device comprising:
an elongate aiming body having two end openings connected to one another by a guiding channel defining an axis for longitudinal guiding of the perforating instrument,
said aiming body being extended at one of the ends by a fork overlapping the transverse process supported by the pedicle to perforate,
the overlapping fork comprising two extensions defining a receiving area for the transverse process supported by the pedicle to be perforated, said receiving area having a first contact surface, configured to engage with an area at the junction of the transverse process and an articular process associated with the pedicle to be perforated, placed offset relative to the guiding axis.

13. The location device according to claim 12, wherein the overlapping fork comprises a portion connecting the aiming body and the extensions being arranged to determine a second contact surface able to enter into contact with the articular process when the first contact surface is in contact with the area at the junction of the process and the articular process.

14. The location device according to claim 13, wherein the guiding axis of the guiding channel is parallel to the longitudinal axis of the aiming body.

15. The location device according to claim 13, wherein the receiving area of the transverse process extends parallel to the guiding axis of the guiding channel.

16. The location device according to claim 15, wherein the receiving area of the transverse process is of a U shape with the base forming the first contact surface.

17. The location device according to claim 16, wherein the aiming body comprises, at an end opposite an end supporting the overlapping fork, an arm extending along a longitudinal axis cutting the guiding axis.

18. The location device according to claim 17, wherein the longitudinal axis of the arm, the guiding axis of the guiding channel and the longitudinal axis of the aiming body are coplanar.

19. The location device according to claim 18, wherein the longitudinal axis of the arm has an angular distance from the guiding axis ranging from 90 degrees to 150 degrees.

20. The location device according to claim 19, wherein the arm is organised to allow for the connection of a handle that can be removed from the aiming body.

21. The location device according to claim 19, wherein the arm forms a handle for the aiming body.

22. The location device according to claim 21, wherein the aiming body and the overlapping fork are made of a single unit.
